(19) United States
(12) Patent Application Publication
(10) Pub. No.: US 2005/0153419 A1
(43) Pub. Date: Jul. 14, 2005

(54) Methods for producing cell lines stable in serum-free medium suspension culture

(75) Inventors: Zhong Liu, Colts Neck, NJ (US); Robert L. Longley JR., North Brunswick, NJ (US); Marc Peter Santoro, Yardley, PA (US); Marcio Voloch, Boston, MA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/019,137

(22) Filed: Dec. 21, 2004

Related U.S. Application Data
(60) Provisional application No. 60/532,275, filed on Dec. 23, 2003.

(51) Int. Cl. C12N 15/861; C12N 7/01; C12N 5/06

(52) U.S. Cl. 435/235.1; 435/325; 435/456

(57) ABSTRACT

The present invention provides methods for adapting cells, such as A549 cells, to growth in serum-free and animal material-free medium suspension culture. The present invention provides methods for preparing viruses, such as adenovirus, from the A549 cells adapted for growth in serum-free and animal material-free medium in suspension culture.

### CROSS-REFERENCE TO RELATED APPLICATIONS

This application claims the benefit of U.S. Provisional Patent Application No. 60/532,275, filed Dec. 23, 2003 which is herein incorporated by reference in its entirety.

### FIELD OF THE INVENTION

The present invention relates to methods for growing cells in culture and the production of virus using the cells.

### BACKGROUND OF THE INVENTION

There are two major barriers in the development of a suspension process for the production of viral vectors. One is the difficulty of maintaining long term culture of the cell inoculum. The second is the tendency towards significantly reduced viral productivity once the production cells are kept in the suspension environment.

Methods for adaptation of A549 cells to serum-free medium in stationary culture are known in the art. For example, in Siegfried et al., (Siegfried, et al., (1994) J. Biol. Chem. 269 (11): 8596-8603), the A549 cell line was adapted to serum-free medium in stationary culture. In this method, A549 cells were first adapted to basal Eagle's medium containing 1% fetal bovine serum over a period of one month. Near confluent monolayers of these A549 cells were washed with saline and placed in a serum-free medium, called Rₒ medium, which was RPMI 1640 phenol red-free supplemented with selenium (30 nM) and glutamine (2 mM). During the adaptation to Rₒ medium, which took approximately one month, colonies emerged that survived without serum, and eventually formed a mixture of attached cells and cells that floated in clusters. Cells adapted to serum and growth-factor free medium were designated A549-Rₒ. The A549-Rₒ cells were propagated for over two years in the absence of any serum or added growth factors. The A549-Rₒ cells were maintained at high cell density (5×10⁵ cells/ml) and were subcultured 1:2 every 14 days. The A549-Rₒ cells had a doubling time of eight to ten days and the parental A549 cells had a doubling time of 30 hours. The A549-Rₒ cells grew at a much slower rate than the parental A549 cells, existed as a mixture of attached cells and cells that floated in large cell clumps or clusters, grew in stationary culture and required a high cell density for optimal growth.

The A549 cell line has historically been propagated as an adherent culture or a stationary culture for the production of viral vectors. The present invention provides novel methods for producing viral vectors in A549 suspension culture.

### SUMMARY OF THE INVENTION

The present invention provides an adapted A549 cell line stable in serum-free and animal material-free medium suspension culture. In one embodiment of the invention, the adapted A549 cell line has the characteristics of the cell line identified as American Type Culture Collection (ATCC) accession number PTA-5708. In another embodiment of the invention, the adapted A549 cell line is the cell line identified as American Type Culture Collection (ATCC) accession number PTA-5708.

The present invention also provides a method for adapting A549 cells to serum-free and animal material-free medium suspension culture comprising the steps of (a) weaning the cells from serum-containing medium to a medium with a final serum concentration from 2.5% to below 1.25% (e.g. from 1.25% to 0%) in adherent culture; (b) introducing the cells to suspension culture; (c) monitoring cell aggregation (e.g., the number of cells per aggregate; the degree of cell aggregation; the distribution of sizes of the cell aggregates); (d) removing cell aggregates; and (e) continuing weaning of the cells in suspension culture to a medium with no serum and/or any other component of animal origin. The A549 cells used for the adaptation method (i.e., the parental cells) may be ATCC strain CCL-185.

The present invention includes a method for producing an adapted A549 cell line stable in serum-free and animal material-free medium suspension culture comprising the steps of (a) weaning the cells from serum-containing medium to a medium with a final serum concentration from 2.5% to below 1.25% (e.g. from 1.25% to 0%) in adherent culture; (b) introducing the cells to suspension culture; (c) monitoring cell aggregation (e.g., the number of cells per aggregate; the degree of cell aggregation; the distribution of sizes of the cell aggregates); (d) removing cell aggregates; (e) continuing weaning of the cells in suspension culture to a medium with no serum; and (f) culturing the cells in serum-free and animal material-free medium suspension culture. Furthermore, the method may include cryopreserving the cells after either step (e) or step (f). In yet another embodiment, the cryopreserved cell line is frozen under either serum-free and animal material-free medium conditions or under serum-containing medium conditions. The method may also comprise storing the cells at temperatures of 0° C. or less.

The present invention provides a method for producing a virus comprising the steps of (a) culturing A549 cells of an adapted A549 cell line stable in serum-free and animal material-free medium suspension culture; (b) inoculating the cells with the virus (e.g., adenovirus, such as CRAV); and (c) incubating the inoculated cells. The method may also comprise the step of exchanging the culture medium with fresh medium after step (a) and before step (b). The method may also comprise the step of adding calcium chloride to the culture and/or exchanging the culture medium with fresh medium with or without the additional calcium chloride (e.g., by perfusion), after step (b). The method may also comprise the step of freezing the cells after step (c). Furthermore, the method may comprise the step of harvesting the virus after step (c). The method may comprise harvesting the virus from the cells and the medium.

In one embodiment of the invention, the adapted A549 cell line exhibits sustained growth and stable viral productivity for at least 137 generations in serum-free and animal material-free suspension culture. In another embodiment of the invention, the adapted A549 cell line has sustained growth and stable viral productivity for at least 6 months in serum-free and animal material-free medium suspension culture.

In one embodiment of the invention, the virus is an adenovirus. In another embodiment, the adenovirus is a conditionally replicating adenovirus. In yet another embodiment, the virus is a recombinant virus. In another embodiment, the recombinant virus carries a heterologous gene.

In one embodiment of the invention, the A549 cell concentration of the adapted A549 cell line stable in serum-free and animal material-free suspension culture at inoculation of the adenovirus is from 1.8×10⁶ cells/ml to 2.4×10⁶ cells/ml. In another embodiment, the A549 cells of the adapted A549 cell line stable in serum-free and animal material-free medium suspension culture are from a culture in the late exponential phase of growth at inoculation of the adenovirus. In another embodiment, the amount of adenovirus inoculated is 1×10⁸ viral particles/ml culture. In yet another embodiment of the present invention, the ratio of adenovirus particles to A549 cells, at inoculation is (40 to 60):1.

In one embodiment of the invention, the A549 cells, of the adapted A549 cell line stable in serum-free and animal material-free medium suspension culture, for the method for producing virus are from a cryopreserved cell line. In yet another embodiment, the cryopreserved cell line is frozen under either serum-free and animal material-free medium conditions or under serum-containing medium conditions.

The scope of the present invention also provides a method for producing adenovirus comprising the steps of (a) weaning A549 cells in a cell line from serum-containing medium (e.g., containing 10% serum (e.g., fetal bovine serum)) to a medium with a final serum concentration from 2.5% to below 1.25% (e.g., from 1.25% to 0%) in adherent culture; (b) introducing the cells to suspension culture; (c) monitoring cell aggregation in the culture (e.g., the number of cells per aggregate; the sizes of the aggregates; the degree of cell aggregation; the distribution of sizes of the cell aggregates) (d) removing cell aggregates; (e) further weaning the cells in suspension culture to a medium with no serum and/or any component of animal origin; (f) concentrating the cells; (g) exchanging the medium to a medium supplemented with a cryoprotectant; (h) freezing the cells (e.g., cryopreserving the cells); (i) storing the cells at a temperature of 0° C. or less; (j) reconstituting the cells to serum-free and animal material-free medium suspension culture; (k) propagating the cells to late exponential phase of growth; (l) exchanging the culture medium with fresh medium (e.g., serum-free and animal material-free medium); (m) inoculating the cells with adenovirus; (n) adding calcium chloride to the culture; (o) incubating the inoculated cells; (p) exchanging the culture medium with fresh medium (e.g., serum-free and animal material-free medium); (q) adding calcium chloride to the culture; (r) incubating the cells; and (s) harvesting the adenovirus. Steps (f)-0), (1), (n), (p), (q) and (s) are optional.

### DETAILED DESCRIPTION OF THE INVENTION

The present invention provides an economical and easy method for producing adenovirus (e.g., in suspension A549 cells) without the problems associated with growth of infected cells in the presence of serum or other medium components of animal origin.

### Generation of an Adapted A549 Cell Line

The present invention includes a method for adapting an A549 cell line for growth in the absence of serum and substances derived from components of animal origin to generate a cell line which exhibits sustained growth in suspension culture and a stable viral production rate when infected with adenovirus. Generally, the A549 cells are adapted by (a) gradually weaning the cells from the serum-containing medium (e.g., medium containing 10% serum) to a medium with a final serum concentration from 2.5% to 1.25%, or from 2.5% to 0.6%, or from 2.5% to 0.5%, or from 2.5% to 0.4%, or from 2.5% to 0.3%, or from 2.5% to 0.2%, or from 2.5% to 0.1%, or from 2.5% to 0.05%, or from 2.5% to 0, in adherent culture or stationary culture; (b) placing the cells in a shaken, rocked, agitated or stirred vessel for suspension culture; (c) measuring cell aggregation or monitoring the degree of cell aggregation in the culture; (d) removing cell aggregates (by any method known in the art); and (e) continuing weaning of the cells in suspension culture to a medium with no serum or any other medium component of animal origin. Preferably, the cells are shaken, rocked, agitated or stirred continuously through steps (b), (c), and (e). In general, the adaptation process takes three to six weeks to complete.

Typically, the adapted cells are stable for at least 137 generations or 6 months in serum-free and animal material-free medium suspension culture (i.e., the cells exhibit sustained growth in serum-free and animal material-free medium suspension culture and a stable viral production rate). Also, the adapted cells have a doubling time in serum-free and animal material-free medium suspension culture that is in the range of 0.8 to 2.9 times the doubling time of the parental A549 cells in stationary culture in serum-containing medium. For example, typically, the doubling time for the adapted A549 cells in serum-free and animal material-free medium is in the range of 24 to 88 hours and the doubling time for the parental A549 cells in serum-containing medium and stationary culture is 30 hours. In the adapted A549 cell line serum-free and animal material-free medium suspension culture, the total cell population is in suspension. In one embodiment greater than 99% of the adapted A549 cells are in suspension (e.g., 100% of the cells are in suspension, 100% of the cells are not attached to a surface, 100% of the cells are suspended in the liquid medium).

In one embodiment of the invention, the adapted A549 cell line has the characteristics of the cell line identified as American Type Culture Collection (ATCC) accession number PTA-5708 which is also called the A549S cell line. Cells of the A549S cell line are stable for at least 137 generations or 6 months in serum-free and animal material-free medium suspension culture (i.e., the cells exhibit sustained growth in serum-free and animal material-free medium suspension culture and a stable viral production rate). The doubling time of the cells of the A549S cell line in serum-free and animal material-free medium suspension culture is in the range of approximately 24 to 88 hours. In the A549S cell line serum-free and animal material-free medium suspension culture, the total A549S cell population is in suspension. In one embodiment greater than 99% of the A549S cells are in suspension (e.g., 100% of the cells are in suspension, 100% of the cells are not attached to a surface, 100% of the cells are suspended in the liquid medium).

In one embodiment of the invention, the adapted A549 cell line is the cell line identified as American Type Culture Collection (ATCC) accession number PTA-5708 which is also called the A549S cell line.

“A549” is a lung carcinoma cell line which is commonly known in the art. In one embodiment, the A549 parental cell line used for the adaptation method is ATCC strain CCL-185.

As used herein, the term “confluent” indicates that the cells have formed a coherent layer on the growth surface where all the cells are in contact with other cells, so that virtually all the available surface is used. For example, “confluent” has been defined (R. I. Freshney, Culture of Animal Cells-A Manual of Basic Techniques, Second Edition, Wiley-Liss, Inc. New York, N.Y., 1987, p. 363) as the situation where “all cells are in contact all around their periphery with other cells and no available substrate is left uncovered”. For the purposes of the present invention, the term “substantially confluent” indicates that the cells are in general contact on the surface even though interstices may remain, such that over 70%, preferably over 90%, of the available surface is used. Here, “available surface” means sufficient surface area to accommodate a cell. Thus, small interstices between adjacent cells that cannot accommodate an additional cell do not constitute “available surface”.

Mammalian cells may be adapted from growth in serum conditions to serum-free conditions by gradually weaning the cells from serum or by direct adaptation. The gradual weaning method may be less stressful for the cultures and may cause less growth lag. The direct adaptation method is quicker, but it is relatively harsh and initial cell densities and viabilities often decrease.

Many cell lines may be directly subcultured from medium containing serum to a serum-free medium. For example, when a culture grown in the presence of serum is in mid-log phase of growth with at least 90% viability, it may be diluted at a 1:2 or 1:3 ratio into serum-free medium. This process is repeated twice weekly until consistent growth is obtained. Initially cultures are inoculated at a higher seeding density than what is normally used for subculturing due to significant loss of cells when directly seeded from serum-supplemented to serum-free medium. The cell growth rate is usually slower in serum-free medium for the first several passages before returning to the rates observed for cells in serum-supplemented medium. If this procedure is not successful, the sequential or weaning method should be used.

“Weaning” cells or a “sequential adaptation” from a serum and serum protein containing medium to a serum and animal material-free medium refers to a gradual, step-wise reduction of the serum concentrations of the medium. The gradual reduction may be done by methods which are well known in the art. For example, cells in a first medium containing a high concentration of serum may be used to inoculate a second medium containing slightly less serum. Once the cells in the second medium have grown to a given cell density, they may, in turn, be used to inoculate a third medium containing even less serum. This process may be repeated until the cells are growing in a medium containing the desired amount of serum.

In another example of weaning cells, the cells are grown in a basal medium supplemented with 10% serum until the cells reach the peak of the linear log phase of growth. Then, the cells are subcultured into serum-free medium base supplemented with 5% serum. The cells are subcultured upon reaching saturation density into serum-free medium base supplemented with 1% serum. Subsequently, at each subculture, reduce the serum by 50% until the serum concentration is below 0.06%. Then, maintain and culture the cells in a serum-free medium. If cell growth declines at any point during the adaptation, return the serum concentration to that promoting the cell growth. Allow the cell growth to stabilize at that serum concentration before proceeding with the serum reduction schedule. Once the cells are adapted to the serum-free conditions, proceed with medium protein reduction schedule, such as, at each subculture add a equal volume of serum-free and animal material-free medium until the culture is propagated under serum-free and animal material-free medium conditions. In a variation of this method, the cells may be adapted directly to serum-free and animal material-free medium conditions, without using the intermediary serum-free medium step.

Another example of weaning cells, is to propagate the cells to a 90% saturation density in serum-containing medium, such as basal medium containing 5-10% serum. Subculture at a 1:1 ratio using 50% serum-containing medium and 50% serum-free medium. The next day, subculture the cells in the same manner. At some point, the cell doubling will decrease and the time interval between cell cultures will increase. Continue to subculture the cells 1:1 as necessary, until such time that the cells are subcultured on a daily basis. Once the cells are adapted to the serum-free conditions, proceed with medium protein reduction schedule, such as, at each 1:1 subculture using 50% serum-free medium and 50% serum-free and animal material-free medium until, such time that the cells are subcultured on a daily basis. At this point, the cells may be adjusted to a subculturing program with a split ratio of greater than 1:1. In a variation of this method, the cells may be adapted directly to serum-free and animal material-free medium conditions, without using the intermediary serum-free medium step.

In another example of weaning cells, at each passage the culture is diluted into a mixture of the serum-containing medium and the serum-free medium. Initially a 1:1 ratio of the serum-containing medium to serum-free medium may be used. With each subsequent passage, the relative amount of the serum-free medium is increased until complete independence of serum is achieved. At each passage, the culture should be in mid-log phase of growth and the dilution into medium should be roughly a 1:2 to 1:3 ratio. Cells should be subcultured twice per week. At each passage, a back-up flask should be seeded with a serum concentration known to be adequate to maintain cell viability in the event that the new medium condition does not succeed.

For cell lines, such as A549, which are adherent in the presence of serum, adaptation to serum-free media or serum-free and animal material-free media will often result in the cultures becoming loosely adherent, possibly with clumping, and with large cell aggregates.

The introduction of cells to suspension culture may be done by methods which are well known in the art. For example, the cells of an adherent culture may be removed from their growth surface using a cell scraper and then placed in a vessel, such as a shake flask or a spinner flask, in which the culture is constantly agitated. In another example, the cells of a culture may be removed from the growth surface by trypsinization, followed by the inactivation of the trypsin, or by removal of the trypsin by washing the cells, and then placing the cells in suspension culture in a vessel. Alternatively, cells cultured in adherent culture may be dislodged from substratum by non-enzymatic procedures, such as by gentle tapping of the culture vessel or by treatment with solutions containing divalent ion chelators. For example divalent ion chelators, such as ethylenediaminetetraacetic acid (EDTA) and ethylene glycol-bis(β-aminoethyl ether)-N,N,N′,N′-tetraacetic acid (EGTA), may be used. The suspension culture may be shaken, rocked, agitated, rolled or stirred to maintain the cells in suspension.

Many cell types tend to grow as cell clumps in suspension culture, especially a culture originally derived from an attached or an adherent cell line. Cultures with varying levels of cell aggregation may display different growth kinetics. The control of aggregate size is an important issue. Cell death and necrosis may occur within aggregates. Severe aggregation may result in poor cell growth as a result of limitations in space and metabolic diffusion. Furthermore, if the cells are host cells for a viral production process, extreme cell aggregation may negatively affect infection efficiency by preventing interior cells of the aggregate from being infected and thereby reducing the overall viral titer obtained. Both biomass measurement and aggregation quantification are important in determining cell growth and behavior in an aggregated suspension culture. Assessment of the degree of cell aggregation in a suspension culture is important for monitoring a suspension process.

The presence of cell aggregates or clumps in the culture may be determined by any method known in the art. For example, the presence of aggregates may be visualized microscopically or by use of a cell sizing apparatus such as a COULTER COUNTER (Beckman Coulter, Inc., Particle Characterization, 1950 West 8th Avenue, Hialeah, Fla., 33010, USA) or an AccuSizer 780/SPOS Single Particle Optical Sizer (Particle Sizing Systems, 668 Woodbourne Road, Suite 104, Langhorne, Pa., 19047, USA). Other automated methods for quantitating cell aggregation are known in the art (Neelamegham et al., Ann. Biomed. Eng. 25(1): 180-9 (1997); Tsao et al., Biotechnol. Prog. 16: 809-814 (2000)). In one embodiment of the invention, the method of Tsao et al. (Biotech. Prog. 16: 809-814 (2000)) may be employed to quantitatively monitor cell aggregation and cell biomass.

The adapted A549 cells are suspension competent cells that grow in serum-free and suspension culture in a mixture of single suspension cells with small aggregates, i.e., cells that are monodisperse and cells in aggregates of sizes of 400 microns in diameter to 20 microns in diameter. The adapted A549 cells have been made competent to growth in serum-free and animal material-free medium suspension culture by gradual adaptation of attachment-dependent cells to those conditions. The amount of cell clumping may also be reduced by adding a lipid mixture to the culture. Addition of a chemically defined lipid mixture may avoid the introduction of animal products to the culture.

During suspension adaptation of A549 cells, cells not associated with large cell clumps may be selectively retained. The selective retention of cells not associated with large cell clumps may be done by methods which are well known in the art. For example, the agitation of the suspension culture is stopped for 1 to 2 minutes allowing large cell aggregates to settle to the bottom of the culture vessel. 90% of the volume of the culture, which contains individual cells and cells in small aggregates, is drawn off and subcultured in a new vessel. The remaining culture volume containing large cell aggregates in 10% of the volume of the original culture is discarded. In another example, the agitation of the suspension culture is stopped for 1 to 2 minutes allowing large cell aggregates to settle to the bottom of the culture vessel. 10% of the volume of the culture, which contains the large cell aggregates, is drawn off with a pipet from the bottom of the vessel and discarded. The remaining culture volume that contains individual cells and small cell aggregates in 90% of the original culture volume is subcultured. Culture vessels of 250 ml, 500 ml and 1 L size shake flasks, preferably have a culture volume of 30 to 40 ml, 100 ml, and 240 ml, respectively.

In this manner, aggregates consisting of a few hundred cells or more are eliminated from the culture e.g., cell population. The desired cell population may be enriched by multiple rounds of selection e.g., by repeating the procedure. The resulting cells will exhibit less clumping or less of a degree of cell aggregation than the non-adapted cells in the same suspension culture medium.

The degree of culture clumping or aggregation during culturing may be monitored by particle, i.e., cell aggregate, size measurement using an AccuSizer 780/SPOS Single Particle Optical Sizer. In this instrument, individual particles are passed by a laser beam and the amount of light blocked by each particle is measured. The amount of light blocked corresponds to the cross sectional area of the particle and thus the cell clump or cell aggregate size. The distribution profile of single cells and cell clumps is reported in a tabular form or as a histogram. The optical sizer is able to detect particle sizes ranging from individual cells e.g., 10 to 15 microns in diameter, to cell aggregates up to 400 microns in diameter. For example, a preferred probe used with the instrument detects particles with a range in sizes of 0.5 microns to 400 microns in diameter.

In one embodiment, the monitoring of cell aggregation or the degree of cell aggregation is performed by the method disclosed in Tsao et al. (Biotechnol. Prog. 16: 809-814 (2000)).

Cells of the adapted A549 cell line may exist in serum-free and animal material-free medium suspension culture as a mixture of single cells with small cell aggregates. This is achieved in part by selectively eliminating large cell clumps or large cell aggregates. It is believed that the cell population that forms larger aggregates has been removed during the course of adaptation. A cell aggregate or cell clump that is removed may be greater than 400 microns in diameter. The cell aggregates remaining and cultured are preferably small, in the range 100 microns to 20 microns in size. The single cell sizes are in the range of 10 to 15 microns in diameter.

Cell aggregates or cell clumps present in the adapted A549, also named A549S, culture stable in serum-free and animal material-free suspension culture may be less than 400 microns in diameter e.g., 350 microns, generally at least 300 microns e.g., 250 microns, at least 200 microns e.g., 150 microns, at least 100 microns e.g., 90, 80, 70, 60 microns, at least 50 microns e.g., 40, 30 microns, and at least 25 microns in diameter. Single cells have a diameter in the range of 10 to 15 microns.

Distribution of particle sizes provides information about the aggregation state of the culture simultaneously with a cumulative cell volume. Quantification of aggregation state using the AccuSizer 780/SPOS Single Particle Optical Sizer is described by the following methods. One method is a histogram summarizing the distribution of cumulative volume of all particles i.e., cells and cell aggregates. The degree of cell clumping may be represented by a cumulative aggregation plot e.g., the cumulative cell volume profile. The description of aggregation may also be presented in a numerical manner. The percentage points are chosen at which the cumulative curves cross the 25%, 50% and 75% marks on the histogram or chart. The numerical presentation of the results, such as the 50% mark, provides a convenient and consistent comparison of the degree of aggregation between samples.

The adapted A549 cell line was deposited under the Budapest Treaty, on Dec. 23, 2003 with the American Type Culture Collection (ATCC), 10801 University Blvd., Manassas, Va., 20110-2209, USA, under the indicated name and accession number as follows; Deposit name: “A549S”; ATCC Accession Number: PTA-5708. All restrictions on access to the cell line deposited with the ATCC will be removed upon grant of a patent.

By “suspension culture” is meant cell culture in which the majority or all of cells in a culture vessel are present in suspension e.g., are not attached to any substratum or surface, the vessel surface, or to another surface within the vessel. The suspension culture may be shaken, rocked, agitated, rolled or stirred to maintain the cells in suspension.

“Serum-containing medium” includes any growth medium containing serum from any organism. For example, serum-containing medium includes media containing fetal bovine sera, newborn calf sera, calf sera, human sera, horse sera, chicken sera, goat sera, porcine sera, rabbit sera, and/or sheep sera. Sera may be heat inactivated, dialyzed, γ-irradiated, delipidated or defibrinated. The sera may also be supplemented, for example, with iron or growth factors.

“Serum-free medium” includes any medium lacking serum. In the art, serum-free media may describe a class of media that do not require supplementation with serum to support cell growth. Serum-free media may contain discrete proteins or bulk protein fractions. The proteins may be animal-derived. Examples of preferred commercially available serum-free media formulations are EX-CELL™ 520 and EX-CELL™ 301, from JRH Biosciences, Inc., 13804 W. 107^{th} Street, Lenexa, Kans., 66215, USA.

“Serum-free and animal material-free” culture media refer to culture media that contain no animal-derived components. In the art, cell culture media manufacturer's definitions of serum-free and serum-free and animal material-free media may vary. A serum-free or a serum-free and animal material-free medium may also be described as a serum-free, chemically-defined medium. These media are a subclass of serum-free media that contain no components of unknown composition. These media are free of animal-derived components and all components have a known chemical structure. Protein-free media are a subclass of serum-free media that are free of all proteins, but may contain plant or yeast hydrolysates.

“Serum-free and animal material-free medium suspension culture” or “serum-free and animal material-free suspension culture” means a suspension culture that is propagated in serum-free and animal material-free medium. The serum-free and animal material-free medium suspension culture comprises cells and medium. The culture contains proteins that are secreted by, derived from, or produced by the cells grown or cultured in the medium. If virus is propagated, the culture comprises cells, virus and medium. A culture producing virus contains proteins that are from the cells and the virus.

Commercially available animal material-free synthetic cell culture medium may be used as the serum-free and animal material-free medium. An example of a preferred serum-free and animal material-free medium includes IS 293-V™ from Irvine Scientific, 2511 Daimler Street, Santa Ana, Calif., 92705, USA.

Commercially available serum-free media may be screened for suitability as the serum-free medium. For example, commercially available media may be screened for their ability to support A549 cell growth in shaker flasks. For example, cells from an adherent culture may be transferred into suspension using the medium of interest. In another example, cells from an already established suspension culture may be switched from their current medium to the medium of interest. Cell growth is monitored by hemacytometer counting. The degree of cell clumping is evaluated by microscopic examination. For example, results from this screening method found that the media, EX-CELL™ 520 and EX-CELL™ 301, from JRH Biosciences, Inc., (13804 W. 107^{th} Street, Lenexa, Kans., 66215, USA) support A549 cell growth without large aggregates. These media may be developed further as a serum-free media. Additional results from the screening method found, for example, that the serum-free and animal material-free medium disclosed in Condon et al., (Biotechnol. Prog. 19: 137-143 (2003)) for suspension culture of HEK293 cells e.g., IS 293-V (Irvine Scientific) supplemented with 0.1% PLURONIC F-68 (GIBCO), 10 mM Tris*HCl (pH 7.4, Biowhittaker), 1× Trace Elements A, B, and C (Mediatech), and 13.4 mg/L ferrous gluconate (Fluka)) supported A549 cell growth without large aggregates. This medium may be developed further as a serum-free and animal material-free medium.

Also, for example, the following commercially available media did not support A549 cell growth in the screening method; CD 293 (GIBCO, Invitrogen); AIM-V® (GIBCO, Invitrogen, Inc.,); RPMI 1640 (GIBCO, Invitrogen, Inc.,); 293 SFM II (GIBCO, Invitrogen, Inc.,); Gene Therapy Medium 3 for Adenovirus Production (Sigma-Aldrich, P.O. Box 14508, St. Louis, Mo., 63178, USA); and CHO Protein-free Medium, Animal Component-free Medium for Suspension Culture (PF-ACF-CHO) (Sigma-Aldrich). These media were not developed further. In addition, for example, cultured A549 cells formed large aggregates in the following commercially available media; ULTRACHO™ (Biowhittaker, Cambrex Corp., One Meadowland Plaza, East Rutherford, N.J., 07073, USA); ULTRACULTURE™ Culture (Biowhittaker, Cambrex Corp.); and IS-CHO-V™ (Irvine Scientific). These media were not developed further.

The serum-free and animal material-free medium is supplemented with an iron supplement designed to replace transferrin for iron transport. An example of a commercially available iron supplement is the Chemically-Defined Iron Supplement from Sigma-Aldrich, P.O. Box 14508, St. Louis, Mo., 63178, USA, product number 13153, that contains 222-334 parts per million (ppm) of iron and a synthetic transport molecule to which the iron binds. This complex is transported into cells where the iron is released and becomes available to the cell. Sigma-Aldrich's Chemically-Defined Iron Supplement is used at a dilution of 1 ml per liter of medium. A preferred example of a commercially available iron supplement is Irvine Scientific's (Irvine Scientific, 2511 Daimler Street, Santa Ana, Calif., 92705, USA) Iron Chelate, product number 9343, used at dilution of 1 ml to 3 ml per liter of medium, preferably 3 ml per liter of medium. Another preferred example of a commercially available iron supplement is ferrous gluconate used at a concentration of 13 mg per liter of medium.

The medium is supplemented with lipids and lipid precursors such as choline, oleic acid, linoleic acid, ethanolamine, or phosphoethanolamine to facilitate the growth of cells. There are commercially available concentrated lipid mixtures that may be utilized to supplement the medium. One example of a commercially available lipid mixture concentrate is Sigma-Aldrich's (Sigma-Aldrich, P.O. Box 14508, St. Louis, Mo., 63178, USA) Lipid Medium Supplement (100×), product number L2273, used at a dilution of 10 ml per liter of medium. The formulation of Sigma-Aldrich's Lipid Medium Supplement (100×) is as follows: 100 ml/L of Sigma-Aldrich's Lipid Mixture, product number L 5146, and 100 g/L PLURONIC F-28, product number P 1300. The formulation of Sigma-Aldrich's Lipid Mixture, product number L 5146, that is used to make the Lipid Medium Supplement (100×), is as follows: cholesterol (4.5 g/L); cod liver oil fatty acids, methyl esters (10 g/L); polyoxyethylenesorbitan monooleate (25 g/L); and D-alpha-tocopherol acetate (2 g/L). A preferred example of a commercially available lipid mixture concentrate is GIBCO, Invitrogen Corporation's, (Invitrogen Corporation, 1600 Faraday Avenue, Carlsbad, Calif., 92008, USA) Chemically Defined Lipid Concentrate, product number 11905-031, used at a dilution of 1 ml to 10 ml per liter of medium e.g., 0.1% v/v to 1% v/v, preferably at 1 ml per liter of medium e.g., 0.1% v/v, more preferably at 4 ml per liter of medium e.g., 0.4% v/v, and even more preferably at 10 ml per liter of medium e.g., 1% v/v. The formulation for GIBCO, Invitrogen Corporation's Chemically Defined Lipid Concentrate, product number 11905-031, is as follows: PLURONIC F-68 (100,000 mg/L); ethyl alcohol (100,000 mg/L); cholesterol (220 mg/L); Tween 80 (also called polyoxyethylenesorbitan monooleate) (2,200 mg/L); DL-alpha-tocopherol acetate (70 mg/L); stearic acid (10 mg/L); myristic acid (10 mg/L); oleic acid (10 mg/L); linoleic acid (10 mg/L); palmitic acid (10 mg/L); palmitoleic acid (10 mg/L); arachidonic acid (2 mg/L); and linolenic acid (10 mg/L).

The serum-free and animal material-free medium is supplemented with a nonionic surface-active agent or a nonionic surfactant, such as, for example, PLURONIC F68. The PLURONICS are a series of nonionic surfactants with the general structure HO(CH₂CH₂O)ₐ(CH(CH₃)CH₂OH)_{b}(CH₂CH₂O)CH where b is at least 15 and (CH₂CH₂O)_{a+c} is varied from 20% to 90% by weight. The PLURONICS are also known, for example, as poloxamers; methyl oxirane polymers, polymer with oxirane; and polyethylenepolypropylene glycols, polymers. A particularly preferred nonionic surfactant is PLURONIC F68. The amount of the nonionic surfactant, such as PLURONIC F68, used may range between 0.05% and 0.4.%, particularly preferred is between 0.1% and 0.05%, more particularly preferred is 0.1%, in the medium. This agent is generally used to protect the cells from the negative effects of agitation and aeration (Murhammer and Goochee, 1990, Biotechnol. Prog. 6: 142-148; Papoutsakis, 1991, Trends Biotechnol. 9: 316-324).

Furthermore, the medium is supplemented with inorganic trace elements to enhance the growth of cells, such as selenium, glutamine, cupric sulfate, ferric citrate, sodium selenite, zinc sulfate, ammonium molybdate, ammonium vanadate, manganese sulfate, nickel sulfate, sodium silicate, stannous chloride, aluminum chloride, barium acetate, cadmium chloride, chromic chloride, cobalt dichloride, germanium dioxide, potassium bromide, silver nitrate, sodium fluoride and zirconyl chloride.

There are commercially available concentrated mixtures of trace elements such as, for example, Mediatech's Trace Elements A: 1,000× Solution, product number 99-182-CI; Mediatech's Trace Elements B: 1,000× Solution, product number 99-175-CI; and Mediatech's Trace Elements C: 1,000× Solution, product number 99-176-CI (Mediatech, Inc., 13884 Park Center Road, Herndon, Va., 20171, USA). Each of the Mediatech's Trace Elements A, Trace Elements B, and Trace Elements C solutions are used at a dilution of 1 ml per liter of medium. The formulation of Mediatech's Trace Elements A: 1,000× Solution, product number 99-182-CI, is as follows: CuSO₄.5H₂O (1.6 mg/L); ZnSO₄.7H₂O (863 mg/L); selenite.2Na (17.3 mg/L); and ferric citrate (1155.1 mg/L). The formulation of Mediatech's Trace Elements B: 1,000× Solution, product number 99-175-CI, is as follows: MnSO₄.H₂O (0.17 mg/L); Na₂SiO₃.9H₂O (140 mg/L); molybdic acid, ammonium salt (1.24 mg/L); NH₄VO₃ (0.65 mg/L); NiSO₄.6H₂O (0.13 mg/L); and SnCl₂ (anhydrous) (0.12 mg/L). The formulation of Mediatech's Trace Elements C: 1,000× Solution, product number 99-176-CI, is as follows: AlCl₃.6H₂O (1.2 mg/L); AgNO₃ (0.17 mg/L); Ba(C₂H₃O₂)₂ (2.55 mg/L); KBr (0.12 mg/L); CdCl₂ (2.28 mg/L); CoCl₂.6H₂O (2.38 mg/L); CrCl₃ (anhydrous)(0.32 mg/L); NaF (4.2 mg/L); GeO₂ (0.53 mg/L); KI (0.17 mg/L); RbCl (1.21 mg/L); and ZrOCl₂.8H₂O (3.22 mg/L).

Additionally, the serum-free and animal material-free medium is supplemented with buffers which help to control the pH levels of the cell cultures. For example, buffers include sodium bicarbonate, monobasic and dibasic phosphate salts, HEPES ((N-2-hydroxyethyl piperazine-N′-(2-enthanesulfonic acid); 4-(2-hydroxyethyl)piperazine-1-ethanesulfonic acid); and salts thereof)), and Tris ((tris(hydroxymethyl)aminomethane; tris(2-aminoethyl)amine; and salts thereof)).

Additionally, the serum-free and animal material-free medium is supplemented with the amino acid, L-glutamine, at a concentration of 2 mM to 20 mM, preferably at least 2 mM e.g., 1 mM or 3 mM, more preferably at least 4 mM e.g., 5 mM, 6 mM, or 7 mM, more preferably at least 8 mM e.g., 9 mM or 101 mM, in the medium.

Optionally, the serum-free and animal material-free medium may be supplemented with a carbohydrate such as D-glucose at a concentration of 0.1 to 10 grams per liter of medium, at least 2 grams per liter of medium.

In one embodiment, the serum-free and animal material-free medium is Irvine Scientific's IS 293-V™ (Irvine Scientific, 2511 Daimler Street, Santa Ana, Calif., 92705, USA), supplemented with 0.1% PLURONIC F68 (Invitrogen Corporation, 1600 Faraday Avenue, Carlsbad, Calif., 92008, USA), Irvine Scientific's Iron Chelate (3 ml per liter of medium), 15 mM TRIS buffer, Mediatech's (Mediatech, Inc., 13884 Park Center Road, Herndon, Va., 20171, USA) Trace Elements A (1 ml per liter of medium), Mediatech's Trace Elements B (1 ml per liter of medium), and Mediatech's Trace Elements C (1 ml per liter of medium), 8 mM L-glutamine, and GIBCO, Invitrogen's Chemically Defined Lipid Concentrate (1% v/v) (Invitrogen Corporation).

In another embodiment, the serum-free and animal material-free medium is Irvine Scientific's IS 293-V™ (Irvine Scientific, Santa Ana, Calif., USA), supplemented with 0.1% PLURONIC F68 (Invitrogen Corporation), ferrous gluconate (13 mg per liter of medium), 15 mM TRIS buffer, Mediatech's Trace Elements A (1 ml per liter of medium), Mediatech's Trace Elements B (1 ml per liter of medium), and Mediatech's Trace Elements C (1 ml per liter of medium), 8 mM L-glutamine, and GIBCO, Invitrogen's Chemically Defined Lipid Concentrate (1% v/v) (Invitrogen Corporation).

### Cell Culture and Virus Production

Adapted A549 cell lines of the invention may be propagated simply by culturing the cells in an appropriate medium, such as a serum-free and animal material-free medium, preferably in a suspension culture.

Once the cells have been adapted, they may be cryopreserved and stored for future use. Preferably, the cells are cryopreserved by propagating the adapted A549 cells to late exponential phase of growth; concentrating the cells; exchanging the growth medium to a medium e.g., serum-free and animal material-free medium or a serum-containing medium, supplemented with a cryoprotectant and a stabilizer; freezing the cells; and storing the cells at a temperature of 0° C. or less.

Preferably, the cells are stored at −70° C. or less e.g., −80° C., or in liquid nitrogen or in the vapor phase of liquid nitrogen.

The cells may be concentrated by any method known in the art. For example, the cells may be concentrated by centrifugation, sedimentation, concentration with a perfusion device (e.g., a sieve) or by filtration. Preferably, the cells are concentrated to at least 1×10⁷ cells/ml.

The cells may be stored in any cryoprotectant known in the art. For example, the cryoprotectant may be dimethyl sulfoxide (DMSO) or glycerol. The cells may be stored in any stabilizer known in the art. For example, the stabilizer may be methyl cellulose or serum.

Prior to freezing down, the concentrated cells may be portioned into several separate containers to create a cell bank. The cells may be stored, for example, in a glass or plastic vial or tube or in a cell culture bag. When the cells are needed for future use, a portion of the cryopreserved cells (from one container) may be selected from the cell bank, thawed and used in serum-free and animal material-free medium suspension culture without adaptation.

Adapted A549 cells may be propagated or grown by any method known in the art for mammalian cell suspension culture. The adapted A549 cells may be grown in serum-free and animal material-free suspension culture without further adaptation. Propagation may be done by a single step or a multiple step procedure. In a single step propagation procedure, the adapted A549 cells are removed from storage and inoculated directly to a culture vessel where production of virus is going to take place. In a multiple step propagation procedure, the adapted A549 cells are removed from storage and propagated through a number of culture vessels of gradually increasing size until reaching the final culture vessel where the production is going to take place. During the propagation steps, the cells are grown under conditions that are optimized for growth. Culture conditions, such as temperature, pH, dissolved oxygen level and the like are those known to be optimal for the particular cell line and will be apparent to the skilled person or artisan within this field (see e.g., Animal Cell culture: A Practical Approach 2^{nd} edition, Rickwood, D. and Hames, B. D. eds., Oxford University Press, New York (1992)).

When propagating adapted A549 cells or adapted A549 cells producing virus e.g., adenovirus, in the cells, the cells may be grown in serum-free or serum-free and animal material-free medium from the original vial to the biomass. The biomass, having high cell density, may be maintained in serum-free or serum-free and animal material-free medium during virus propagation and production process.

Adapted A549 cells may be grown and the adapted A549 cells producing virus may be cultured in any suitable vessel which is known in the art. For example, cells may be grown and the infected cells may be cultured in a biogenerator or a bioreactor. Generally, “biogenerator” or “bioreactor” means a culture tank, generally made of stainless steel or glass, with a volume of 0.5 liter or greater, comprising an agitation system, a device for injecting a stream of CO₂ gas and an oxygenation device. Typically, it is equipped with probes measuring the internal parameters of the biogenerator, such as the pH, the dissolved oxygen, the temperature, the tank pressure or certain physicochemical parameters of the culture (for instance the consumption of glucose or of glutamine or the production of lactate and ammonium ions). The pH, oxygen, and temperature probes are connected to a bioprocessor which permanently regulates these parameters. In other embodiments, the vessel is a spinner flask, a roller bottle, a shaker flask or in a flask with a stir bar providing mechanical agitation. In another embodiment, a the vessel is a WAVE Bioreactor (WAVE Biotech, Bridgewater, N.J., U.S.A.). The suspension culture may be shaken, rocked, agitated, rolled or stirred to maintain the cells in suspension.

Cell density in an adapted A549 culture may be determined by any method known in the art. For example, cell density may be determined microscopically e.g., hemacytometer, or by an electronic cell counting device (e.g., COULTER COUNTER; AccuSizer 780/SPOS Single Particle Optical Sizer).

The term “generation number” refers to the number of population doublings that a cell culture has undergone. The calculation of population doublings is well known in the art (see, e.g., Patterson, Methods in Enzymology, eds. Jakoby and Pastan, Academic, New York, 58: 150-151 (1979)). In one embodiment, the in vitro cell age or generation number of a culture is determined by calculating the number of cell divisions during the culture period, following the formula, ln(fold of increase in cell mass)/ln2. In one embodiment, the increase in cell mass is measured by the method disclosed in Tsao et al. (Biotechnol. Prog. 16: 809-814 (2000)).

The term “recombinant” refers to a genome which has been modified through conventional recombinant DNA techniques.

The term “virus” as used herein includes not only naturally occurring viruses but also recombinant viruses, attenuated viruses, vaccine strains, and so on. Recombinant viruses include, but are not limited to, viral vectors comprising a heterologous gene. The term recombinant virus includes chimeric (or even multimeric) viruses, i.e. vectors constructed using complementary coding sequences from more that one viral subtype. See, e.g., Feng et al. Nature Biotechnology 15: 866-870 (1997). In some embodiments, helper function(s) for replication of the viruses is provided by the host cell, a helper virus, or a helper plasmid. Representative vectors include, but are not limited to, those that will infect mammalian cells, especially human cells, and may be derived from viruses such as retroviruses, adenoviruses, adeno-associated viruses, herpesviruses, and avipox viruses.

Any virus may be propagated in the cell cultures of the present invention. In one embodiment, the virus is adenovirus. The term “adenovirus” is synonymous with the term “adenoviral vector” and refers to viruses of the genus adenoviridiae. The term adenoviridae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof. For example, any of adenovirus types 1, 2, 3, 4, 4a, 5, 6, 7, 7a, 7d, 8, 9, 10, 11 (Ad11A and Ad11P), 12, 13, 14, 15, 16, 17, 18, 19, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91 may be produced in a cell culture of the invention. In the preferred practice of the invention, the adenovirus is or is derived from the human adenovirus serotypes 2 or 5.

In one embodiment of the invention, the adenovirus comprises a wild-type, unmutated genome. In another embodiment, the virus comprises a mutated genome; for example the mutated genome may be lacking a segment or may include one or more additional, heterologous genes. In another embodiment, the virus is a selectively replicating recombinant virus or a conditionally replicating virus, i.e., a virus that is attenuated in normal cells while maintaining virus replication in tumor cells, see, e.g., Kim, D. et al., Nat. Med. 7: 781-787 (2001); Alemany, R. et al. Nature Biotechnology 18: 723-727 (2000); Ramachandra, M. et al., Replicating Adenoviral Vectors for Cancer Therapy in Pharmaceutical Delivery Systems, Marcel Dekker Inc., New York, pp. 321-343 (2003).

In one embodiment of the invention, the selectively replicating recombinant virus is a selectively replicating recombinant adenovirus or an adenoviral vector such as those described in published international application numbers, WO 00/22136 and WO 00/22137; Ramachandra, M. et al., Nature Biotechnol. 19: 1035-1041 (2001); Howe et al., Mol. Ther. 2(5): 485-95 (2000); and Demers, G. et al. Cancer Research 63: 4003-4008 (2003).

A selectively replicating recombinant adenovirus may also be described as, but not limited to, an “oncolytic adenovirus”, an “oncolytic replicating adenovirus”, a “replicating adenoviral vector”, a “conditionally replicating adenoviral vector” or a “CRAV”.

In another embodiment of the invention, the adenovirus is 01/PEME, also known as cK9TB or K9TB, that is modified to attenuate replication in normal cells by deletions in the E1a gene and the E3 region, insertion of a p53 responsive promoter driving an E2F antagonist, E2F-Rb, and insertion of a major later promoter regulated E3-11.6K gene and is described, for example, in Ramachandra, M. et al., Nature Biotechnol. 19: 1035-1041 (2001); United States Patent Application Publication Number U.S. 2002/0150557; and Demers, G. et al. Cancer Research 63: 4003-4008 (2003).

The term “infecting” means exposing the virus to the adapted A549 cells under conditions to facilitate the infection of the cells with the virus. In cells which have been infected by multiple copies of a given virus, the activities necessary for viral replication and virion packaging are cooperative. Thus, it is preferred that conditions be adjusted such that there is a significant probability that the adapted A549 cells are multiply infected with the virus. An example of a condition that enhances the production of virus in the adapted A549 cells is an increased virus concentration compared to the cell concentration in the infection phase. However, it is possible that the total number of infections per cell may be too high, resulting in toxic effects to the cells. Consequently, it is preferable to maintain the ratio of virus particles to A549 cells, at infection to (40 to 60):1.

The term “culturing under conditions to permit replication of the viral genome” means maintaining the conditions for the infected A549 cells so as to permit the virus to propagate. Virus-containing cells include cells infected by the virus and cells producing virus. It is desirable to control culture conditions so as to maximize the number of viral particles produced by each cell. It is desirable to monitor and control culture conditions such as temperature, dissolved oxygen, pH, agitation, among other parameters known to the skilled artisan. Commercially available bioreactors such as the BIOSTAT line of bioreactors (B. Braun Biotech, Inc., Allentown, Pa., USA) have provisions for monitoring and maintaining such parameters. The optimization of infection and culture conditions will vary somewhat, however, conditions for the efficient replication and production of virus may be achieved by those of skill in the art taking into consideration, for example, the known properties of the cell line, properties of the virus and the type of bioreactor.

Virus, such as adenovirus, may be produced in the adapted A549 cells or suspension A549 cells of the invention. Virus may be produced by culturing the adapted A549 cells; optionally adding fresh growth medium to the cells; inoculating the cells with the virus; optionally supplementing the cell culture with calcium chloride (CaCl₂); incubating the inoculated cells (for any period of time); optionally adding fresh growth medium to the inoculated cells; optionally supplementing the cell culture with calcium chloride; and optionally harvesting the virus from the cells and the medium. Typically, when the concentration of viral particles, as determined by conventional methods, such as high performance liquid chromatography using a Resource Q column, as described in Shabram, et al. Human Gene Therapy 8: 453-465 (1997), begins to plateau, the harvest is performed.

Typically, the infected, adapted A549 cells are capable of maintaining production of the CRAV adenovirus in the range of 36×10⁹ to 144×10⁹ vp/ml for at least 137 generations or at least 6 months in culture.

Fresh medium may be provided to the cells before and/or after virus inoculation. For example, the fresh medium may be added by perfusion. Medium exchange increases the level of virus production in the adapted A549 cells or in the adapted A549 cultures. In one embodiment of the invention, the medium of infected adapted A549 cells is subject to two consecutive exchanges, one exchange upon infection and another exchange one day post-infection. Fresh medium may be provided to the cells with or without additional calcium.

Calcium may be provided to the adapted A549 cells after virus inoculation. The calcium is added to the culture in a soluble form, for example, as calcium chloride or calcium sulfate. Calcium addition increases the level of virus production in the adapted A549 cells or in the adapted A549 cultures. In one embodiment of the invention, calcium chloride is added to the culture after virus infection. In another embodiment, calcium chloride is added two hours after virus inoculation. In another embodiment, calcium chloride is added to the culture in the range of two to eight hours after virus infection. In another embodiment, calcium chloride is added in the range of twenty to twenty four hours after infection. The range of additional calcium chloride concentrations used in the fresh medium or in the cell culture is from 0.2 mM to 1.6 mM. In one embodiment of the invention, the infected adapted A549 cells or the infected adapted A549 cell culture is subject to two consecutive exchanges of fresh medium supplemented with an additional 1.6 mM calcium chloride, one exchange upon infection and another exchange one day post-infection.

The adapted A549 cells used to produce the virus may be derived from a cell line frozen under serum-free and animal material-free medium conditions or from a cell line frozen under serum-containing medium conditions e.g., from a frozen cell bank.

Suitable methods for identifying the presence of the virus in the culture, i.e., demonstrating the presence of viral proteins in the culture, include immunofluorescence tests, which may use a monoclonal antibody against one of the viral proteins or polyclonal antibodies (Von Bülow et al., in Diseases of Poultry, 10^{th} edition, Iowa State University Press), polymerase chain reaction (PCR) or nested PCR (Soine et al., Avian Diseases 37: 467-476 (1993)), ELISA (Von Bülow et al., in Diseases of Poultry, 10^{th} edition, Iowa State University Press)), hexon expression analyzed by flow cytometry (Musco et al. Cytometry 33: 290-296 (1998), virus neutralization, or any of the common histochemical methods of identifying specific viral proteins.

Titrating the quantity of the virus in the culture may be performed by techniques known in the art, as described in Villegas et al., “Titration of Biological Suspensions,” In: A Laboratory Manual for the Isolation and Identification of Avian Pathogens, 3^{rd} Ed., Purchase et al., Eds., Kendall/Hunt Publishing Co., Dubuque, Iowa (1989). In a particular embodiment, the concentration of viral particles is determined by the Resource Q assay as described by Shabram, et al. Human Gene Therapy 8: 453-465 (1997). As used herein, the term “lysis” refers to the rupture of the virus-containing cells. Lysis may be achieved by a variety of means well known in the art. For example, mammalian cells may be lysed under low pressure (100-200 psi differential pressure) conditions, by homogenization, by microfluidization, or by conventional freeze-thaw methods.

The virus-containing cells may be frozen. Virus may be harvested from the virus-containing cells and the medium. In one embodiment, the virus is harvested from both the virus-containing cells and the medium simultaneously. In a particular embodiment, the virus producing cells and medium are subjected to cross-flow microfiltration, as described in U.S. Pat. No. 6,146,891, under conditions to both simultaneously lyse virus-containing cells and clarify the medium of cell debris which would otherwise interfere with virus purification.

Virus may be harvested from the virus-containing cells and medium separately. The virus-containing cells may be collected separately from the medium by conventional methods such as differential centrifugation. Harvested cells may be stored frozen or further processed by lysis to liberate the virus. Virus may be harvested from the medium by chromatographic means. Exogenase free DNA/RNA may be removed by degradation with DNAse/RNAse, such as BENZONASE (American International Chemicals, Inc.).

The virus harvest may be further processed to concentrate the virus by methods such as ultrafiltration or tangential flow filtration as described in U.S. Pat. Nos. 6,146,891 and 6,544,769.

Viral particles produced in the cell cultures of the present invention may be isolated and purified by any method which is commonly known in the art. For example, the viral particles may be purified by cesium chloride gradient purification, column or batch chromatography, diethylaminoethyl (DEAE) chromatography (Haruna et al. Virology 13: 264-267 (1961); Klemperer et al., Virology 9: 536-545 (1959); Philipson Virology 10: 459-465 (1960)), hydroxyapatite chromatography (U.S. Patent Application Publication Number U.S. 2002/0064860) and chromatography using other resins such as homogeneous cross-linked polysaccharides, which include soft gels (e.g., agarose), macroporous polymers based on synthetic polymers, which include perfusion chromatography resins with large “throughpores”, “tentacular” sorbents, which have tentacles that were designed for faster interactions with proteins (e.g., fractogel) and materials based on a soft gel in a rigid shell, which exploit the high capacity of soft gels and the rigidity of composite materials (e.g., Ceramic HyperD® F) (Boschetti, Chromatogr. 658: 207 (1994); Rodriguez, J. Chromatogr. 699: 47-61 (1997)). In a particular embodiment, the virus is purified by column chromatography, for example, as described in Huyghe et al. Human Gene Therapy 6: 1403-1416 (1995); U.S. Pat. No. 5,837,520; and U.S. Pat. No. 6,261,823.

### Protein Purification

Proteins produced by adenoviruses grown in the adapted A549 cells of the invention, preferably adenovirus comprising a heterologous gene encoding a polypeptide of interest, may also be isolated and purified.

The proteins, polypeptides and antigenic fragments of this invention may be purified by standard methods, including, but not limited to, salt or alcohol precipitation, affinity, preparative disc-gel electrophoresis, isoelectric focusing, high pressure liquid chromatography (HPLC), reversed-phase HPLC, gel filtration, cation and anion exchange and partition chromatography, and countercurrent distribution. Such purification methods are well known in the art and are disclosed, e.g., in “Guide to Protein Purification”, Methods in Enzymology, Vol. 182, M. Deutscher, Ed., 1990, Academic Press, New York, N.Y.

### EXAMPLES

The following examples are provided to more clearly describe the present invention and should not be construed to limit the scope of the invention in any way.

Table 1 lists various media used in the examples.

TABLE 1
Media

| Medium | | |
|---|---|---|
| Identifier | Purpose | Composition |
| Medium 1 | Adherent cell growth | Dulbecco's modified Eagle's |
| | | medium (DMEM)/High glucose |
| | | supplemented with 4 mM |
| | | L-glutamine and 10% gamma- |
| | | irradiated characterized |
| | | fetal bovine serum. |
| Medium 2 | Adaptation to serum- | Irvine Scientific's IS 293-V ™; |
| | free and animal | supplemented with 0.1% |
| | material-free medium | PLURONIC F-68; 15 mM Tris, |
| | suspension cell growth; | 13 mg/L ferrous gluconate; |
| | Serum-free and animal | 1× Mediatech's Trace Elements |
| | material-free medium | A (1 ml per liter of medium); |
| | suspension cell growth | 1× Mediatech's Trace Elements |
| | and virus production | B (1 ml per liter of medium); |
| | | 1× Mediatech's Trace Elements |
| | | C; 8 mM L-glutamine; Gibco, |
| | | Invitrogen's Chemically |
| | | Defined Lipid Concentrate |
| | | (10 ml per liter of medium). |
| Medium 3 | Adaptation to serum- | Irvine Scientific's IS 293-V ™; |
| | free and animal | supplemented with 0.1% |
| | material-free medium | PLURONIC F-68; 15 mM Tris; |
| | suspension cell growth; | Irvine Scientific's Iron Chelate |
| | Serum-free and animal | (3 ml per liter of medium); |
| | material-free medium | 1× Mediatech's Trace Elements |
| | suspension cell growth | A (1 ml per liter of medium); |
| | and virus production | 1× Mediatech's Trace Elements |
| | | B (1 ml per liter of medium); |
| | | 1× Mediatech's Trace Elements |
| | | C; 8 mM L-glutamine; Gibco, |
| | | Invitrogen's Chemically |
| | | Defined Lipid Concentrate |
| | | (10 ml per liter of medium). |
| Medium 4 | Cryopreservation | 90% Medium 2, 10% dimethyl |
| | | sulfoxide (DMSO) and 0.1% |
| | | methyl cellulose. |
| Medium 5 | Cryopreservation | 80% Medium 2, 10% DMSO and |
| | | 10% gamma-irradiated |
| | | characterized fetal bovine |
| | | serum. |

### Example 1

### Adaptation of Adherent A549 Cells into Serum-Free and Animal Material-Free Medium Suspension Culture

Following standard protocols for culturing adherent cells by trypsinization, A549 cells were thawed and passaged in Medium 1 (Table 1) in T-75 culture flasks. The adaptation process takes three to six weeks to complete. To initiate the process of suspension adaptation, the attached cells were gradually weaned from serum by serial passages of the cells through medium containing progressively lower levels of serum. This was done by diluting Medium 1 (see Table 1) with increasing volumes of serum-free and animal material-free suspension medium, Medium 2 (see Table 1), at each cell culture passage. As a result, serum levels were decreased stepwise, from the original 10% fetal bovine serum (FBS) level by 50% at each passage to a final FBS concentration below 0.3%. Each passage takes three to five days. The cells were passaged until some the cells became non-adherent, (e.g. are not attached to the surface of the culture vessel).

After one passage in 0.3% FBS containing medium, the cells were trypsinized from the T75 flask, reseeded into a 250 ml shaker flask (40 ml culture volume) in the same 0.3% FBS containing medium, and grown in a shaker incubater at a temperature of 37° C., with an atmosphere of 5% CO₂ and shaking at 85 rpm.

Upon transfer to the suspension culture, all subsequent subculturing was performed with the serum-free and animal material-free medium, Medium 2 (see Table 1), to complete the weaning from serum. Cells were allowed to grow to approximately 2×10⁶ to 2.5×10⁶ cells/ml. The culture was then split 1:2 with Medium 2 (see Table 1) into a 500 ml shaker flask (100 ml culture volume). Cells were allowed again to grow to approximately 2×10⁶ to 2.5×10⁶ cells/ml before being split 1:2 into a IL shaker flask (240 ml culture volume). The culture viability was maintained above 90% as determined by staining with trypan blue.

Cell growth and aggregation were monitored daily using a particle sizer, an AccuSizer 780/SPOS Single Particle Optical Sizer. For the aggregation profile of the culture, a 50% reading of less than or equal to 100 cells/clump gives the best growth rate. Culture viability was measured using trypan blue dye exclusion and a hemacytometer. Monitoring the cell aggregate size permitted the determination of culture conditions, such as the effect of medium modifications and agitation rate, for optimal cell growth through control of cell aggregation. Duplicate cultures were made and one parameter was changed for the culture conditions of one of the duplicate cultures (such as agitation speed) and the degree of aggregation was monitored over time using the particle sizer. In addition, particle size measurements were continuously performed to determine subculturing schedules. The particle sizer gives a reading of cell mass which is equivalent to cell density and maintenance of aggregation within desired parameters. The cell mass reading was used to determine when to split the culture as well as the split ratio. For the aggregation profile, the maintenance of a 50% reading of less than or equal to 100 cells/clump gave the best growth rate.

For continuous propagation of the culture in IL flasks, cells were continually monitored using the particle sizer and subcultured as described above. Particle sizer analysis showed that A549 cells tended to form large aggregates during the first few passages in suspension culture. Large aggregates were allowed to settle to the bottom of the shaker flask by stopping the agitation for one to two minutes before subculturing so that the aggregates could be eliminated from the population through pipeting. Cultures were subcultured in this manner until aggregation was reduced to desirable levels. A desirable level is one in which there are no large clumps that settle to the bottom of the culture flask after 1 to 2 minutes and a 50% cell reading using the particle sizer that is less than or equal to 100 cells/clump. The culture growth rate was maintained. The growth rate observed is at least 0.3 day⁻¹. Cells adapted to suspension growth in serum-free and animal material-free medium may be referred to as “suspension A549 cells” or “adapted A549 cells” or “A549S” or “ATCC accession number PTA-5708”.

TABLE 2
Details of an adaptation of A549 cells to serum-free
and animal material-free medium suspension culture.

| Time | |
|---|---|
| (Days from | |
| thaw of vial) | Observations and actions |
| 0 | 1.2. One vial of the A549 cells were thawed into |
| | Medium 1. |
| | 1.2.1. One T-75 flask contains one fourth of the cells |
| | resurrected from the vial. |
| 1 | 1.3. Split the T-75 flask (1.2.1) at 1:3 ratio into |
| | 3 T-75 flasks using trypsinization. |
| | 1.3.1. One T-75 flask contains 50% of the medium used |
| | in 1.2. and 50% of Medium 2. The serum concentration |
| | in the medium was 5%. |
| 4 | 1.4. Split the T-75 flask (1.2) at 1:4 ratio into |
| | 4 T-75 flasks using trypsinization. |
| | 1.4.1. One T-75 flask contains 25% of the medium used |
| | in 1.2 and 75% of Medium 2. The serum concentration |
| | in the medium was 2.5%. |
| 5 | 1.5. Medium exchange on flask |
| | (1.4.1) with 100% of Medium 2. The serum concentration |
| | in the medium was 0. |
| 6 | 1.6. Cells in T-75 (1.5) detached by trypsinization |
| | (1.3) and resuspended into 10 ml of the original |
| | conditioned medium and agitated at 105 rpm in a 125 ml |
| | shaker flask. The serum concentration in the medium |
| | was 0. |

Maintained culture in serum-free and animal material-free medium
suspension culture from this point forward using a range of agitation
conditions of 80 to 105 rpm, relative to shake flask size and condition
of the culture.

| | |
|---|---|
| 8 | 1.7. Culture (1.6) split at a ratio of 1:3 (final 30 ml) |
| | with Medium 2 and transferred the 30 ml culture to |
| | a new 250 ml shaker flask agitated as 1.6. |
| 11 | 1.8. 30 ml of Medium 2 added to the culture (1.7). |
| 14 | 1.9. Day 14: culture (1.8) split at a ratio of 1:3 |
| | (final 30 ml) with Medium 2. |
| 18 | 1.10. Culture (1.9) split at a ratio of 1:4 (final |
| | 30 ml) with Medium 2. |
| 20 | 1.11. Culture (1.10) split at a ratio of 1:3 (final |
| | 30 ml) with Medium 2. |
| 25 | 1.12. Culture (1.11) split at a ratio of 1:3 with |
| | Medium 2. |
| 28 | 1.13. Culture (1.12) transferred to new 250 ml shake |
| | flasks to remove the cells adhered to vessel wall. |
| 28 | 1.14. 80% medium exchange of culture (1.13) with |
| | Medium 2 (final 13-14 ml). |
| 32 | 1.15. 1:2 split of culture (1.14) with Medium 2 |
| | (final ˜22 ml) |
| 34 | 1.16. 1:2 split of culture (1.15) with Medium 2. |
| 36 | 1.17. Split culture (1.16) to 0.4 × 10⁶ cells/ml |
| | with Medium 2. |
| 39 | 1.18. 10 ml of culture (1.17) was removed so |
| | that ˜28 ml of the culture remained. |
| 40 | 1.19. 25 ml of Medium 2 (1.17) was added to culture |
| | (1.18). |
| 42 | 1.20. 8 ml of the culture (1.19) was removed for the |
| | preparation of 2 frozen vials (Stock) in Medium 4. |
| | 1.20.1. Frozen vial of the adapted A549 cell line stock. |

TABLE 3
Details of a scale-up of an adapted A549 cell line in order to
make the adapted A549 cell line suspension cell bank #1.

| Time | |
|---|---|
| (Days from | |
| thaw of vial) | Observations and actions |
| 0 | 2.1. One frozen adapted A549 cell line stock vial |
| | (1.20.1) thawed into 2 untreated T-75 flasks in |
| | Medium 2 (20 ml/flask) |
| 3 | 2.2. 15 ml of the culture from one of the T-75 (2.1) |
| | was transferred to a 125 ml shakerflask (agitated at |
| | 80 rpm) with 5 ml of Medium 2 added. |
| 4 | 2.3. 15 ml of the culture (2.2) was transferred to a |
| | 250 ml shaker flask (agitated at 80 rpm) with 15 ml of |
| | Medium 2 added |
| 7 | 2.4. Culture (2.3) was sampled for hemacytometer |
| | measurement |
| 8 | 2.5. Culture (2.3) was split at a ratio of 1:2 by |
| | adding 30 ml Medium 2. |
| 11 | 2.6. 30 ml of the culture (2.5) was transferred to a |
| | 500 ml shaker flask (agitated at 80 rpm) with 30 ml of |
| | Medium 2 added |
| 14 | 2.7. 55 ml of the culture (2.6) was transferred to a |
| | 1000 ml shaker flask (agitated at 80 rpm) with 55 ml of |
| | Medium 2 added |
| 15 | 2.8. 110 ml Medium 2 was added to the culture (2.7). |
| 18 | 2.9. A frozen cell bank (21 vials) of the adapted A549 |
| | cell line was prepared from ˜220 ml of the culture |
| | (2.8) using Medium 5. |

### Example 2

### Comparison of the Amount of Cell Aggregation of A549 Cells from Different Cell Lines in Suspension Culture

During the serum-free and animal material-free medium suspension adaptation of A549 cells to create the adapted A549 suspension cell line, cells which were not associated with large cell clumps were selectively retained. Cells or a subpopulation of the cell line not attached to a surface was selected for and propagated in serum-free and animal material-free medium suspension culture. The desired cell population was enriched by multiple rounds of selection by stopping the agitation of the culture and allowing large cell aggregates to settle to the bottom of the flask and subculturing the cells that stay suspended. The resulting cells of the adapted A549 cell line were less aggregated than the non-adapted A549 cells in the same suspension medium (see, for example, Table 3).

The A549 adherent cells were trypsinized, washed with Medium 1 (see Table 1) once, and then seeded into 125 ml shake flasks, in a 20 ml volume, in either Medium 1 or 2 (see Table 1). Cells were grown for six days in a shaker incubator with a 5% CO₂ atmosphere, at a temperature of 37° C., and an agitation speed of 85 rpm.

TABLE 3
Comparison of cultures derived from different A549 cell lines.

| | | A549 cells derived from an | |
|---|---|---|---|
| | | adherent culture grown in | |
| | A549 cells derived from an | Medium 1, placed in serum-free | Adapted A549 cell line |
| | adherent culture grown in | and animal material-free | in serum-free and |
| | Medium 1 and placed in | medium (Medium 2) suspension | animal material-free |
| | suspension culture using | culture for six days but prior | medium suspension |
| Particle | Medium 1 for six days | to suspension adaptation. | culture (Medium 2) |
| Diameter | Cumulative Volume | Cumulative Volume | Cumulative Volume |
| (microns) | Distribution (%) | Distribution (%) | Distribution (%) |
| 15.00 | 1 | 5 | 47 |
| 30.00 | 20 | 27 | 94 |
| 45.00 | 38 | 40 | 98 |
| 60.00 | 54 | 61 | 99 |
| 75.00 | 65 | 77 | 100 |
| 90.00 | 72 | 86 | 100 |

### Example 3

### Production of CRAV by A549S Cells in Serum-Free and Animal Material-Free Medium Suspension Culture

Viral production by A549S cells was carried out in both Erlenmeyer flasks on an orbital shaker and in a stirred tank bioreactor. In both cases, production was achieved by infecting cultures with a virus inoculum.

For virus production in shaker flasks, the temperature (37° C.), CO₂ level (5%) and humidity were maintained by placing the shaker in a tissue culture incubator. The suspension A549 cells grew to a density of approximately 1.8×10⁶ to 2.4×10⁶ cells/ml prior to infection in serum-free and animal material-free medium, (Medium 2, see Table 1), in batch mode. Before virus inoculation, a medium exchange of approximately 90% of the original culture volume was performed with serum-free and animal material-free medium, (Medium 2, see Table 1), by centrifugation. Virus was inoculated at a final concentration of 1×10⁸ virus particles/ml, the equivalent of an approximately (40 to 50) to 1 ratio of virus particles to cell. Two hours after virus inoculation, calcium chloride was added to the culture to provide an additional 1.6 mM calcium chloride to the culture. Approximately 20 hours post-infection, another 90% medium exchange with the serum-free and animal material-free medium, Medium 2 (see Table 1) supplemented with an additional 1.6 mM CaCl₂, was performed by centrifugation. Three ml of culture sample was collected from each culture at 24 hours, 48 hours and 72 hours post-infection to quantify the amount of virus produced. The amount of virus produced was 100×10⁹ to 150×10⁹ vp/ml or 3×10⁴ to 4×10⁴ vp/cell.

For production in bioreactors, stirred tank bioreactors were fitted with an internal spin filter and equipped with a pitch blade impeller. The culture temperature was maintained at 37° C. with a heating blanket. Dissolved oxygen was maintained at 40% of air saturation. The flow rate of air in the headspace was maintained at 0.1 L/minute. The bioreactor tanks were inoculated with cells from shaker flasks with an initial seeding density of 0.5×10⁶ cells/ml in serum-free and animal material-free medium, (Medium 2, see Table 1). The agitation rate was maintained at 120 rpm during the entire experiment. When the cell density reached approximately 1.8×10⁶ to 2.4×10⁶ cells/ml, a perfusion with 3.8 L of serum-free and animal material-free medium, (Medium 2, see Table 1), was performed. Virus was then inoculated at a final concentration of 1×10⁸ virus particles per ml immediately after the perfusion. As in the case with shaker flasks, additional CaCl₂ (1.6 mM) was added to the culture in the tank two hours post-infection. Approximately 20 hours post-infection, another perfusion with 3.8 liters of serum-free and animal material-free medium, Medium 2 (see Table 1), was performed. The pH was kept above 6.9 post-infection with a 5% Na₂CO₃ solution. The virus titer was measured using a Resource Q column as described in Shabram, et al. Human Gene Therapy 8: 453-465 (1997).

### Example 4

### Stability of the Adapted A549 Cell line in Serum-Free and Animal Material-Free Medium Suspension Culture

The A549S cells were continuously passaged during the test period, for six months, and at predetermined intervals culture aliquots were infected for the evaluation of CRAV productivity. These infection experiments were performed repeatedly in an identical manner throughout the life of the culture. Productivity was evaluated over the in vitro culture age expressed as cell generation numbers.

In general, a production host cell line should be stable over a sufficient number of generations to ensure a scalable process, for example, a minimum of 60 generations. First, the cell culture has to be able to maintain its growth in a chosen culture environment for an extended period of time. Second, the level of production should not drift in a significant manner at the end of a defined culture age. Third, the quality of the production generated at different culture ages should be comparable. To evaluate the stability of the adapted A549 cell line, the changes in the growth rate and virus production rate were monitored. The growth rate was derived by dividing the number of generations (or cell divisions) that take place by the number of days over which that growth takes place (see Table 4). This may also be expressed as ln(fold of increase in cell mass)/(time at end of culture-time at beginning of culture (in days)).

The data indicates that the adapted A549 cells are ready to grow immediately after being resurrected from frozen stock to serum-free and animal material-free medium suspension culture, as shown in the first data point of the growth curve. This translates to 40% cell growth per day. This is followed by a gradual increase in growth rate until reaching an apparent plateau at approximately generation 60. The initial increase in growth rate is common among many cell lines when the culture is initiated from a cryogenically-preserved condition.

The range of average growth rates in the Table 4 data for the A549S cells was from 0.19 to 0.69 (day⁻¹) with an average of the twenty-two data points of 0.42 (day⁻¹). This corresponds to a range in doubling time (hours), calculated from the average growth rate (day⁻¹) with the formula (0.693×24)/average growth rate, of 24 to 88 hours and an average doubling time of 40 hours.

While continuing the culture for the measurement of its growth rate, satellite cultures were split off and were infected with the adenoviral vectors for evaluation of virus production. For the satellite cultures, the A549S cells were allowed to grow to approximately 1.8×10⁶ to 2.4×10⁶ cells/ml prior to infection. Before virus inoculation, a medium exchange of approximately 90% of the original culture volume was performed with fresh culture medium (Medium 2, see Table 1). Virus was inoculated at a final concentration of 1×10⁸ vp/ml. At approximately two hours post-infection, calcium chloride (800 μM) was added to the culture. At approximately 20 hours post-infection, another 90% medium change with growth medium (Medium 2, se Table 1) supplemented with 800 μM calcium chloride was performed. Infected culture samples were collected at 24, 48 and 72 hours post-infection for the quantification of virus produced. The virus titer was measured using a Resource Q column as described in Shabram, et al. Human Gene Therapy 8: 453-465 (1997). The maximum virus titer was achieved at approximately 48 hours post-infection in all cases. The virus productivity is presented as volumetric productivity in Table 4. The range of volumetric viral productivity in Table 4 was from 3.63×10¹⁰ to 1.44×10¹¹ (vp/ml). The average volumetric viral productivity for the twenty-one data points in Table 4 was 8.21×10¹⁰ (vp/ml).

TABLE 4
Stability results of an A549S culture
from an adapted A549 cell line.

| Culture Age | | Volumetric Viral |
|---|---|---|
| (Number of | Average Growth | Productivity |
| Cell Divisions) | Rate (Day⁻¹) | (vp/ml) |
| 11 | 0.39 | 5.27 × 10¹⁰ |
| 14 | 0.35 | |
| 15 | 0.69 | 5.47 × 10¹⁰ |
| 18 | 0.26 | 4.96 × 10¹⁰ |
| 20 | 0.23 | 4.27 × 10¹⁰ |
| 23 | 0.30 | 3.63 × 10¹⁰ |
| 26 | 0.26 | 3.89 × 10¹⁰ |
| 28 | 0.19 | 5.49 × 10¹⁰ |
| 31 | 0.35 | 7.78 × 10¹⁰ |
| 36 | 0.51 | 6.63 × 10¹⁰ |
| 41 | 0.50 | 7.33 × 10¹⁰ |
| 43 | 0.22 | 1.44 × 10¹¹ |
| 53 | 0.46 | 1.27 × 10¹¹ |
| 62 | 0.44 | 1.00 × 10¹¹ |
| 70 | 0.40 | 9.33 × 10¹⁰ |
| 74 | 0.46 | 8.24 × 10¹⁰ |
| 83 | 0.46 | 1.02 × 10¹¹ |
| 89 | 0.51 | 9.14 × 10¹⁰ |
| 101 | 0.63 | 1.39 × 10¹¹ |
| 112 | 0.53 | 1.11 × 10¹¹ |
| 131 | 0.49 | 9.62 × 10¹⁰ |
| 137 | 0.57 | 8.96 × 10¹⁰ |

### Example 5

### Cryopreservation of A549 Suspension Cells

Cryopreservation of A549 suspension cell banks using both serum-containing, (Medium 5, see Table 1) and animal material-free freezing medium (Medium 4, see Table 1) was performed. Cells were cultured as described in Example 1. The standard protocol described in “Culture of Animal Cells”, R.I. Freshney, Wiley & Sons Inc., NY, 2000, pp. 297-308 was followed to prepare the frozen cell banks. In the case of animal material-free banks, the freezing medium used Medium 4 (see Table 1). For serum containing banks, Medium 5 (see Table 1) was used.

Thawed cells from both banks readily grew in suspension without the need for re-adaptation. The growth rates for both banks after thawing were very comparable (see, for example, Table 5). Subsequent virus productivity by the two banks was also unaffected by serum-free cryopreservation (see, for example, Table 6). Vials from the cell banks were thawed in 37° C. water bath, washed once with Medium 2 (see Table 1) by centrifugation, and then seeded into 125 ml shake flask using 20 ml of Medium 2 (see Table 1). Growth rates were calculated as given in Example 4. Infections were performed as described in Example 4 for the satellite cultures.

TABLE 5
Cell growth of A549S cultures from
cryopreserved A549S cell line.

Total Cell Growth after Thawing
(Fold in cumulative cell growth)

| | Serum- | Serum- | Serum- | Serum- |
|---|---|---|---|---|
| Time | containing | containing | free | free |
| (Days) | Bank 1 | Bank 2 | Bank 1 | Bank 2 |
| 2 | 5.0 | 4.2 | 3.8 | 4.0 |
| 4 | 21.0 | 18.4 | 13.6 | 14.9 |
| 8 | 174.7 | 153.6 | 125.4 | 140.8 |
| 11 | 424.3 | 409.0 | 330.2 | 364.8 |
| 14 | 1781.8 | 1605.1 | 1397.8 | 1505.3 |

TABLE 6
Production of virus by A549S cultures
from cryopreserved A549S cell line.
Productivity of CRAV (10⁹ vp/ml)

| Serum-containing | Serum-containing | Serum-free | Serum-free |
|---|---|---|---|
| Bank 1 | Bank 2 | Bank 1 | Bank 2 |
| 110 | 108 | 104 | 113 |

### Example 6

### Comparison of CRAV Production Before and After Suspension Adaptation of A549 Cells

Infections were performed under the same conditions, in serum-containing medium (Medium 1, see Table 1) and in stationary culture dishes, using A549 cells of either the adapted A549 cell line (A549S) or A549 cells from an adherent culture. Infection cultures were performed in duplicate.

The adapted A549 cells from a serum-free and animal material-free medium suspension culture grown in Medium 2 (see Table 1) were seeded into several T-25 culture flasks in Medium 1 (see Table 1) at 80% to 100% of confluence and allowed to attach to the flask surface for 24 hours. A549 cells grown entirely as an adherent culture (non-adapted) were seeded into several T-25 flasks four days before infection and allowed to grow to 80% to 100% confluence in Medium 1 (see Table 1). At the time of infection, cultures from both cell lines were given an exchange of medium using Medium 1 (see Table 1) and were infected with either 1×10⁸ or 4×10⁸ vp/ml using CRAV. Twenty-four hours post-infection, the viral inoculum was removed and replaced with fresh Medium 1 (see Table 1). In addition, one representative flask for each cell line was taken at 24 hours post-infection, trypsinized, and the number of cells per flask was determined by hemacytometer counting and trypan blue staining. At days two and three post-infection, flasks from each cell line were frozen at −80° C. and processed for Resource Q HPLC analysis. The total amount of virus produced by the cultures was divided by the number of cells present at 24 hours post-infection to determine specific productivity for the two cell lines. Infections were performed on the suspension-adapted cells, A549S, in stationary culture dishes using DMEM containing 10% FBS (see Table 1, Medium 1), the formulation used for attached culture. The A549S cells showed no reduction in the level of virus production in comparison with non-adapted, control A549 cells on a per cell basis (see, for example, Table 7).

TABLE 7
Comparison of the specific viral productivities of non-adapted,
adherent A549 cells to A549S cells using infection conditions
of stationary culture with serum-containing medium

| | Day 2 Post-Infection | Day 3 Post-Infection |
|---|---|---|
| | Specific Viral | Specific Viral |
| | Productivity | Productivity |
| Cell type | (10⁴ vp/cell) | (10⁴ vp/cell) |
| Adherent | 9.1 using 1 × 10⁸ vp/ml | 8.5 using 1 × 10⁸ vp/ml |
| A549 cells | 9.8 using 4 × 10⁸ vp/ml | 7.4 using 4 × 10⁸ vp/ml |
| (non-adapted) | | |
| Adapted | 10.7 using 1 × 10⁸ vp/ml | 11.3 using 1 × 10⁸ vp/ml |
| A549 cells | 10.4 using 4 × 10⁸ vp/ml | 10.5 using 4 × 10⁸ vp/ml |
| (A549S) | | |

### Example 7

### Effect of Calcium Chloride Addition on CRAV Production in A549S Cells In Serum-Free and Animal Material-Free Suspension Culture

The effect of calcium chloride addition on CRAV production was evaluated in shake flasks. For virus production in shake flasks, the temperature (37° C.), CO₂ level (5%) and humidity level were maintained by placing the shakers in a tissue culture incubator. The suspension A549S cells grew to a density of approximately 1.8×10⁶ to 2.4×10⁶ cells/ml prior to infection in serum-free and animal material-free medium (Medium 2, see Table 1) in batch mode. Before virus inoculation, a medium exchange of approximately 90% of the original culture volume was performed with serum-free and animal material-free medium (Medium 2, see Table 1) by centrifugation. Virus was inoculated at a final concentration of 1×10⁸ virus particles/ml, the equivalent of an approximately (40 to 50) to 1 ratio of virus particles to cell.

At approximately 2 hours post-virus inoculation (post-infection), calcium chloride solutions were added to the culture to achieve the target calcium chloride (in addition to the amount of calcium already contained in the culture medium) concentration of the 200 μM to 1600 μM, specifically for the following calcium chloride concentrations of 200 μM, 400 μM, 800 μM and 1600 μM. Second, a medium perfusion was performed by centrifugation at approximately 20 hours post-infection with fresh Medium 2 containing same amount of additional calcium chloride as conducted with the calcium chloride addition performed at 2 hours post-infection. A control culture was included in which no calcium chloride was added at 2 hours post-infection or with the fresh Medium 2 (see Table 1) perfusion at 20 hours post-infection. Three ml of culture sample was collected at 48 hours post-infection from each culture to quantify the amount of virus produced. The amount of virus produced was measured by Resource Q HPLC as described in Shabram, et al. Human Gene Therapy 8: 453-465 (1997). The results are shown in Table 8.

TABLE 8
Effect of calcium chloride addition on CRAV production
in A549S cells cultured in serum-free and animal
material-free suspension culture.

| Calcium Chloride | CRAV Titer |
|---|---|
| Addition (μM) | (10⁹ vp/ml) |
| 0 | 78.7 |
| 200 | 84.9 |
| 400 | 82.6 |
| 800 | 102.2 |
| 1600 | 104.7 |
| 3200 | 106.7 |

### Example 8

### Effect of Viral Inoculum Concentration on CRAV Production

The effect of viral inoculum concentration on CRAV production using A549S cells was examined in shake flasks. A549S cells from a frozen bank were thawed and passaged in Medium 2 (see Table 1) until they displayed stable growth. Two one liter shake flask cultures were grown to a concentration of approximately 2.7×10⁶ cells/ml and the cultures combined. A medium exchange of approximately 85% of the original culture volume was performed by centrifugation, and the cells resuspended to a final cell density of approximately 3.6×10⁶ cells/ml and aliquoted into fourteen 125 ml shake flasks. The cells were then inoculated with CRAV virus at concentrations ranging from 0.125×10⁸ vp/ml to 8×10⁸ vp/ml (see Table 9); duplicate infections were performed for each concentration. Inoculated cells were grown at 37° C., 5% CO₂, and high humidity in a tissue culture incubator. At two hours post-infection, calcium chloride was added to each of the cultures to provide an additional 1.6 mM calcium chloride (CaCl₂) to the cultures. Approximately 20 hours post-infection, another 85% medium exchange was performed using Medium 2 (see Table 1) supplemented with 1.6 mM CaCl₂. Three ml samples were collected from each culture at 24, 48, 72, and 96 hours post-infection for the quantification of CRAV virus produced. Table 9 shows that by day 3 or 4 post-infection, there was little difference in virus titer from cultures infected in the range of 0.5×10⁸ vp/ml to 8×10⁸ vp/ml.

TABLE 9
Production of CRAV virus by A549S cultures at
different virus inoculum concentrations; values
are the average of duplicate samples.

| | CRAV Production (10¹⁰ vp/ml) | | | |
|---|---|---|---|---|
| CRAV | Day 1 | Day 2 | Day 3 | Day 4 |
| Inoculum | Post- | Post- | Post- | Post- |
| (vp/ml) | Infection | Infection | Infection | Infection |
| 0.125 × 10⁸ | 0.1 | 5.3 | 7.1 | 6.6 |
| 0.25 × 10⁸ | 0.2 | 7.8 | 9.2 | 8.5 |
| 0.5 × 10⁸ | 0.2 | 9.6 | 9.7 | 9.3 |
| 1 × 10⁸ | 0.3 | 11.4 | 10.4 | 9.8 |
| 2 × 10⁸ | 0.6 | 12.3 | 10.6 | 10.2 |
| 4 × 10⁸ | 0.8 | 11.8 | 9.2 | 9.6 |
| 8 × 10⁸ | 1.0 | 12.2 | 9.5 | 10.0 |

The present invention should not be limited in scope by the specific embodiments described herein. Indeed, various modifications of the invention, in addition to those described herein, will become apparent to those skilled in the art from the foregoing description. Such modifications fall within the scope of the appended claims.

Patents, patent applications, publications, product descriptions and protocols are cited throughout this application, the disclosures of which are incorporated herein by reference in their entireties.

1. An adapted A549 cell line stable in serum-free and animal material-free medium suspension culture.

2. The cell line of claim 1, wherein the adapted A549 cell line is the cell line identified as ATCC accession number PTA-5708.

3. A method for adapting A549 cells to serum-free and animal material-free medium suspension culture comprising the steps of:
(a) weaning the cells from serum-containing medium to a medium with a final serum concentration from 2.5% to below 1.25% in adherent culture;
(b) introducing the cells to suspension culture;
(c) monitoring cell aggregation;
(d) removing cell aggregates; and
(e) continuing weaning of the cells in suspension culture to a medium with no serum.

4. The method of claim 3, wherein the A549 cells are ATCC strain CCL-185.

5. A method of producing an adapted A549 cell line stable in serum-free and animal material-free medium suspension culture comprising the steps of:
(a) adapting A549 cells by the method according to claim 3; and
(b) culturing the cells in serum-free and animal material-free medium suspension culture.

6. The method of claim 5, further comprising storing the cells at temperatures of 0° C. or less.

7. The method of claim 5, further comprising cryopreserving the cells.

8. A method for producing a virus comprising the steps of:
(a) culturing A549 cells of the adapted A549 cell line of claim 1 in serum-free and animal material-free medium suspension culture;
(b) inoculating the cells with the virus; and
(c) incubating the inoculated cells.

9. The method of claim 8, further comprising freezing the cells after step (c).

10. The method of claim 8, further comprising harvesting the virus after step (c).

11. The method of claim 10, wherein the virus is harvested from the cells and the medium.

12. The method of claim 8, wherein the virus is an adenovirus.

13. The method of claim 8, wherein the virus is a recombinant virus.

14. The method of claim 8, wherein the virus carries a heterologous gene.

15. The method of claim 12, wherein the adenovirus is a conditionally replicating adenovirus.

16. The method of claim 8, further comprising adding calcium chloride to the culture, after step (b).

17. The method of claim 8, wherein the A549 cell concentration at inoculation of the virus is from 1.8×10⁶ cells/ml to 2.4×10⁶ cells/ml.

18. The method of claim 12, wherein the amount of adenovirus inoculated is 1×10⁸ viral particles/ml medium.

19. The method of claim 12, wherein the ratio of virus particles to cells at inoculation, is (40 to 60):1.

20. The method of claim 8, further comprising exchanging the culture medium with fresh medium after step (a) and before step (b).

21. The method of claim 8, further comprising after step (c), the steps of (d) exchanging the culture medium with fresh medium; and (e) incubating the cells.

22. The method of claim 8, further comprising exchanging the culture medium with fresh medium after step (a) and before step (b); and after step (c).

23. The method of claim 8, wherein the A549 cells are from a cryopreserved cell line.

24. The method of claim 8, wherein the A549 cells are from a cell line adapted to serum-free and animal material-free medium suspension culture.

25. A method for producing adenovirus comprising the steps of:
(a) weaning A549 cells from serum-containing medium to a medium with a final serum concentration from 2.5% to below 1.25% in adherent culture;
(b) introducing the cells to suspension culture;
(c) monitoring cell aggregation;
(d) removing the cell aggregates;
(e) continuing weaning of the cells in suspension culture to a medium with no serum;
(f) propagating the cells to late exponential phase of growth;
(g) exchanging the culture medium with fresh medium;
(h) inoculating the cells with the adenovirus;
(i) adding calcium chloride to the culture;
(j) incubating the inoculated cells;
(k) exchanging the culture medium with fresh medium;
(l) incubating the cells;
(m) adding calcium chloride to the culture;
(n) incubating the cells; and
(o) harvesting the adenovirus.

26. The method of claim 25 further comprising the steps of:
(i) concentrating the cells;
(ii) exchanging the medium with a medium supplemented with a cryoprotectant;
(iii) freezing the cells;
(iv) storing the cells at a temperature of 0° C. or less; and
(v) reconstituting the cells to serum-free and animal material-free medium suspension culture;
after step (e), but before step (f).
